(19) United States
(12) Patent Application Publication
(10) Pub. No.: US 2017/0203063 A1
(43) Pub. Date: Jul. 20, 2017

(54) VENTILATOR-RESUSCITATOR DEVICE AND METHOD OF VENTILATION

(71) Applicant: Evgeny PECHERER, Netanya (IL)

(72) Inventor: Evgeny PECHERER, Netanya (IL)

(21) Appl. No.: 15/312,653

(22) PCT Filed: May 28, 2015
(86) PCT No.: PCT/IB2015/054030

§ 371 Date: Nov. 20, 2016

Related U.S. Application Data
(60) Provisional application No. 62/003,554, filed on May 28, 2014.

(51) Int. Cl. A61M 16/00; A61M 16/20; A61M 16/06; A61M 16/12

(52) U.S. Cl. CPC A61M 16/0063; A61M 16/125; A61M 16/202; A61M 16/0051; A61M 16/06; A61M 2202/0208; A61M 2205/3331; A61M 2016/0027; A61M 2205/70; A61M 2205/581; A61M 2205/8206; A61M 2240/00

(57) ABSTRACT

A ventilator-resuscitator device comprising a mixing chamber, a compressor, a controllable pressure-relief vent, and a controller, is provided. The mixing chamber has air and oxygen inlets, and an outlet. The compressor is configured to operate crate at two or more power levels and to increase, by a different amount at each power level, the pressure of a gas flowing between a low-pressure side thereof and a high-pressure side thereof, thereby producing a pressurized gas, the low-pressure side being in fluid communication with the outlet of the mixing chamber. The vent comprises an orifice configured to bring the high-pressure side of the compressor into fluid communication with the atmosphere. The controller is configured to direct operation thereof and to regulate the pressure of the pressurized gas by selectively operating the compressor at one of its power levels.

### FIELD OF THE INVENTION

The present disclosure relates to ventilator-resuscitator devices and systems, and especially ventilator-resuscitator devices and system designed for use with neonatal patients.

### BACKGROUND OF THE INVENTION

Ventilator-resuscitator systems are used in medical procedures to deliver a positive pressure to a patient. Often, in neonatal patients (e.g., newborns, including premature infants), a ventilator-resuscitator system is used to deliver a positive pressure to the patient's lungs.

Typically, ventilator-resuscitator systems are used in conjunction with an oxygen source, which may be mixed with atmospheric air to make a gas mixture, which is raised to a suitable pressure for delivery to the patient.

During ventilation, a doctor may vary the pressure of the gas mixture delivered to the patient, thereby simulating breathing in the patient's lungs in an effort to induce him to breathe on his own.

### SUMMARY OF THE INVENTION

According to one aspect of the presently disclosed subject matter, there is provided a ventilator-resuscitator device comprising:

a mixing chamber having an air inlet, an oxygen inlet, and an outlet;

a compressor configured to operate at two or more power levels and to increase, by a different amount at each power level, the pressure of a gas flowing between a low-pressure side thereof and a high-pressure side thereof, thereby producing a pressurized gas, the low-pressure side being in fluid communication with the outlet of the mixing chamber;

a controllable pressure-relief vent comprising an orifice configured to bring the high-pressure side of the compressor into fluid communication with the atmosphere; and

a controller configured to direct operation thereof;

wherein the controller is configured to regulate the pressure of the pressurized gas by selectively operating the compressor at one of its power levels.

It will be appreciated that in the present disclosure and the claims, the term “power level”, when used with reference to operation of the compressor, refers to the degree by which it increases the pressure of a gas flowing therethrough. A change in power level may be associated with a commensurate change in the amount of electrical energy required to drive the compressor, but the compressor may be designed to operate at different power levels without requiring different amount of electrical energy to drive it at each power level without departing from the scope of the present disclosure and the claims.

The controller may be further configured to regulate the pressure of the pressurized gas by operating the controllable pressure-relief vent to selectively change the size of the orifice.

The controller may be configured to regulate the pressure of the pressurized gas by concurrently operating:

the controller at one of its power levels; and

the controllable pressure-relief vent to change the size of the orifice.

The ventilator-resuscitator device may be configured to selectively close the orifice completely.

The controllable pressure-relief vent may comprise one or more solenoids configured to regulate the size of the orifice.

The ventilator-resuscitator device may further comprise a pressure sensor configured to measure the pressure of gas exiting therefrom.

The controller may be configured to perform a calibration based on the pressure measured by the pressure sensor.

The controller may be configured to activate an alarm when an unexpected increase in pressure is measured by the pressure sensor.

The controller may be configured to activate an alarm when an unexpected decrease in pressure is measured by the pressure sensor.

The controller may be configured to cyclically regulate the pressure of the pressurized gas between two pressures at a predetermined rate.

The controller may be configured to emit an audible sound at a predetermined rate.

The compressor may comprise a variable-speed drive.

The ventilator-resuscitator device may comprise a battery configured to supply electrical power necessary for its operation.

The oxygen inlet of the mixing chamber may comprise an oxygen inlet nipple configured for attachment thereto of a flexible tube.

The ventilator-resuscitator device may further comprise an outlet nipple, configured for attachment to a flexible tube, and being in fluid communication with the high-pressure side of the compressor.

The controllable pressure-relief vent may be in fluid communication between the high-pressure side of the compressor and the outlet nipple.

According to another aspect of the presently disclosed subject matter, there is provided ventilator-resuscitator system comprising a ventilator-resuscitator device as described above, and a mask configured to:

be brought into fluid communication with the high-pressure side of the compressor for receipt therefrom of a pressurized gas; and

be fitted to the face of a patient for delivery thereto of the pressurized gas.

According to a further aspect of the presently disclosed subject matter, there is provided a method of ventilating a patient, the method comprising providing a ventilator-resuscitator device comprising:

a mixing chamber for producing an oxygen-air mixture and having an air inlet, an oxygen inlet, and an outlet;

a compressor configured to operate at two or more power levels and to increase, by a different amount at each power level, the pressure of a the oxygen-air mixture flowing between a low-pressure side thereof and a high-pressure side thereof, the low-pressure side being in fluid communication with the outlet of the mixing chamber to receive therefrom the oxygen-air mixture;

a controllable pressure-relief vent comprising an orifice configured to bring the high-pressure side of the compressor into fluid communication with the atmosphere; and

a controller configured to direct operation thereof; the method further comprising:

connecting a mask to the ventilator-resuscitator device to receive the compressed oxygen-air mixture therefrom;

fitting the mask to the patient to deliver the oxygen-air mixture from the ventilator-resuscitator device thereto;

operating the ventilator-resuscitator system to deliver a pressurized oxygen-air mixture to the patient;

regulating the pressure of the oxygen-air mixture between two pressures by selectively operating the compressor at two of its power levels.

The method may further comprise regulating the pressure of the oxygen-air mixture by concurrently operating:

the controller at two of its power levels; and

the controllable pressure-relief vent to change the size of the orifice.

The method may further comprise closing the orifice completely.

The controllable pressure-relief vent may comprise one or more solenoids configured to regulate the size of the orifice.

The ventilator-resuscitator device may further comprise a pressure sensor configured to measure the pressure of gas exiting the ventilator-resuscitator sensor.

The method may further comprise performing a calibration based on the pressure measured by the pressure sensor.

The method may further comprise activating an alarm when an unexpected increase in pressure is measured by the pressure sensor.

The method may further comprise activating an alarm when an unexpected decrease in pressure is measured by the pressure sensor.

The method may further comprise cyclically regulating the pressure of the oxygen-air mixture between two pressures at a predetermined rate.

The compressor may comprise a variable-speed drive.

The ventilator-resuscitator device may comprise a battery configured to supply electrical power necessary for its operation.

The oxygen inlet may comprise an oxygen inlet nipple configured for attachment thereto of a flexible tube.

The ventilator-resuscitator device may comprise an outlet nipple, configured for attachment to a flexible tube connected to the mask, in fluid communication with the high-pressure side of the compressor.

The controllable pressure-relief vent may be located in fluid communication between the high-pressure side of the compressor and the compressor outlet nipple.

The patient may be a neonatal patient.

According to modifications of the above aspects, the ventilator-resuscitator device may be provided without a mixing chamber. Accordingly, it may be designed such that the low-pressure side of the compressor is connected only to an oxygen source, or be designed such that the low-pressure side of the compressor is in fluid communication only with the atmosphere.

### BRIEF DESCRIPTION OF THE DRAWINGS

For a better understanding of the embodiments and to show how it may be carried into effect, reference will now be made, purely by way of example, to the accompanying drawings.

With specific reference now to the drawings in detail, it is stressed that the particulars shown are by way of example and for purposes of illustrative discussion of selected embodiments only, and are presented in the cause of providing what is believed to be the most useful and readily understood description of the principles and conceptual aspects. In this regard, no attempt is made to show structural details in more detail than is necessary for a fundamental understanding; the description taken with the drawings making apparent to those skilled in the art how the several selected embodiments may be put into practice. In the accompanying drawings:

FIG. 1 illustrates as ventilator-resuscitator system according to the presently disclosed subject matter;

FIG. 2 is a top perspective view of a ventilator-resuscitator device of the ventilator-resuscitator system illustrated in FIG. 1, with a housing thereof removed;

FIG. 3 is a bottom perspective view of functional elements of the ventilator-resuscitator device illustrated in FIG. 2;

FIG. 4 is a cross-sectional view of a mixing chamber of the device illustrated in FIG. 2, taken along line in FIG. 3; and

FIG. 5 is a schematic illustration of a fluid path defined by the ventilator-resuscitator device illustrated in FIG. 2.

### DETAILED DESCRIPTION OF EMBODIMENTS

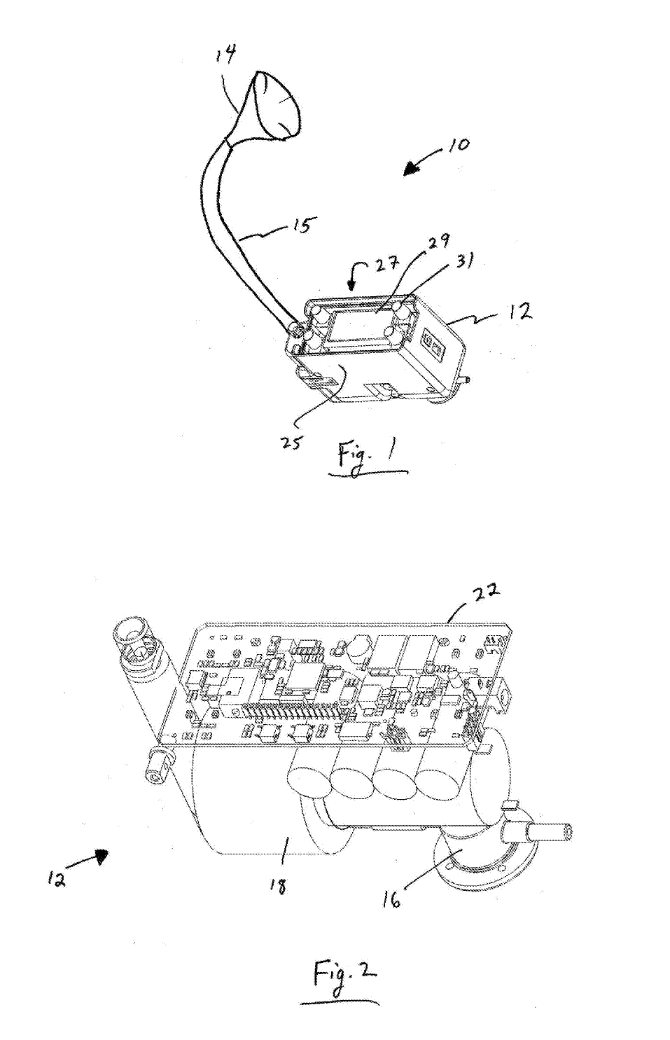

As illustrated in FIG. 1, there is provided a ventilator-resuscitator system, which is generally indicated at 10. The ventilator-resuscitator system 10 may be used, e.g., to stimulate breathing in neonatal patients. It may thus be designed to simulate a desired breathing pattern, for example with respect to pressure of a gas mixture delivered to the patient and/or the pressure thereof.

The ventilator-resuscitator system comprises a ventilator-resuscitator device 12 configured to provide a pressurized gas (e.g., an oxygen-air mixture; herein, the terms “gas”, “gas mixture”, “oxygen-air mixture”, and similar terms, used alone or with one or more qualifiers such as “pressurized”, are used interchangeably, unless otherwise clear from context, and should not be construed to limit the disclosure and/or claims), and a mask 14, connected thereto by a flexible tube 15, for delivery of the pressurized gas from and configured for connection to the ventilator-resuscitator device.

According to some modifications, the ventilator-resuscitator system 10 may comprises a different suitable apparatus in place of the mask 14 in order to deliver the pressurized gas to the patient.

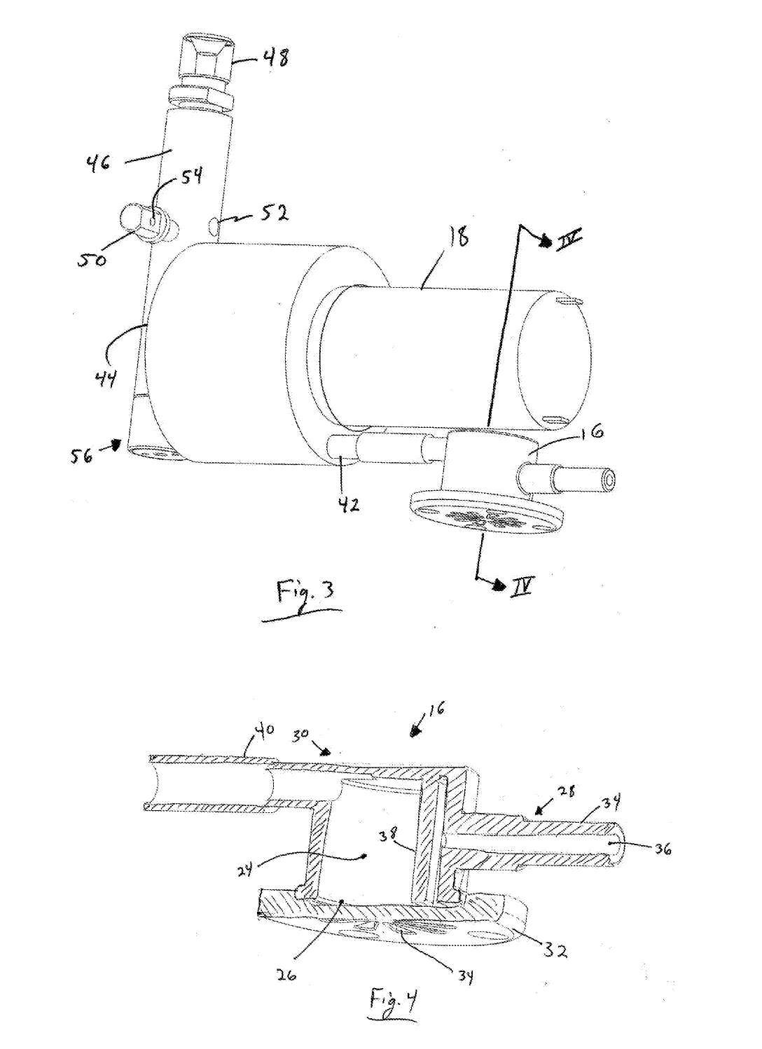

As seen in FIGS. 2 and 3, the ventilator-resuscitator device 12 comprises a mixing chamber 16 for receiving therein and mixing constituent elements of the gas mixture, a compressor 18 for increasing the pressure of the gas mixture, a controllable pressure-relief vent 50 for facilitating regulating the pressure of the pressurized gas mixture, and a controller 22 (not shown in FIG. 3) for directing operation of the ventilator-resuscitator device. Reverting to FIG. 1, it may further comprise a housing 25 containing elements of the ventilator-resuscitator device 12 therewithin, and a user interface 27, which may comprise one or more data-presentation elements 29 (such as a screen, as shown) and one or more input devices 31 (such as knobs, as shown). The input devices 31 are in communication with the controller 22 to allow a doctor to adjust operating parameters of the ventilator-resuscitator device 12, such as minimum and maximum operating pressures, cycle frequency, etc. For example, the maximum pressure may be selected to simulate a peak inspiratory pressure associated with inhalation, and the minimum pressure may be selected to simulate a positive end-expiration pressure associated with the end of exhalation.

It will be appreciated that although the controller 22 is described and illustrated in the present disclosure and the claims as constituting an independent element of the ventilator-resuscitator device 12, it may comprise one or more physical elements.

Furthermore, the ventilator-resuscitator device 12 may be provided such that some functions described herein as being performed by the controller are performed by other elements described herein, and vice-versa, without departing from the scope of the presently-disclosed subject matter and/or claims.

As illustrated in FIG. 4, the mixing chamber 16 comprises a mixing compartment 24 with an air inlet 26, an oxygen inlet 28, and an outlet 30. The air inlet 26 may be open to the atmosphere, and may be provided with a filter lid 32 with through-going apertures 34 allowing atmospheric air to flow therethrough to the mixing compartment 24. A filter (not shown) may be provided between the filter lid 32 and the mixing compartment 24, e.g., to remove impurities from atmospheric air before it is mixed with oxygen to make the mixture.

The oxygen inlet 28 comprises an oxygen inlet nipple 34 configured for attachment thereto of a flexible tube, for example connected to a pressurized oxygen source, and defining therein a path 36 leading to the mixing compartment 24. The mixing compartment 24 may comprise a baffle 38 disposed therewithin and oriented such that oxygen entering therein via the path 36 at an elevated pressure is dissipated, thereby facilitating mixing.

The outlet 30 is in fluid communication with the compressor 18, for example via a connecting tube 40. The operation of the compressor 18 to increase the pressure of the gas creates a negative pressure within the connecting tube 40. The ventilator-resuscitator device 12 may be designed such that the negative pressure is greater than the pressure of the oxygen delivered via the oxygen inlet 28 plus the pressure of atmospheric air, which results in a negative pressure at the air inlet 26 (i.e., a pressure towards the mixing compartment 24). Thus, the ventilator-resuscitator device 12 may be designed such that no additional elements are necessary to draw atmospheric air into the mixing chamber 24, and gas does not backflow from the mixing chamber through the oxygen inlet 28, at least during normal operation thereof.

The compressor 18 may be any suitable element configured to increase the pressure of a gas flowing therethrough. Reverting to FIG. 3, it comprises a low-pressure side 42 constituting an inlet thereof, and a high-pressure side 44 constituting an outlet thereof. It may be configured to operate at two or more power levels, wherein it increases the pressure of gas flowing between the low-pressure side 42 and the high-pressure side 44 by a different amount at each power level. According to some examples, this is accomplished by the compressor 18 comprising variable-speed drive. According to some modifications, by providing a variable-speed drive, the power requirements of the ventilator-resuscitator device 12 are reduced, enabling power for operation thereof to be supplied by a battery (not illustrated).

The high-pressure side 44 of the compressor 18 is in fluid communication with an outlet tube 46. The outlet tube 46 comprises an outlet nipple 48, configured for attachment thereto of the flexible tube 15. The outlet tube 46 comprises a controllable pressure-relief vent 50 and a measurement outlet 52.

The pressure-relief vent 50 comprises an orifice 54 which is in fluid communication with the atmosphere (e.g., it may be open to the interior of the casing 25, which may comprise one or more apertures open to the atmosphere). The orifice 54 may be much smaller than the opening of the outlet nipple 48, thus being operable to lower the pressure of gas exiting from the high-pressure side 44 of the compressor 18, e.g., slightly, without lowering it to atmospheric pressure. The pressure-relief vent 50 may further be configured to selectively change the size of the orifice 54, for example by use of a solenoid (not illustrated) or any other suitable element. According to some examples, the solenoid may be configured to close the orifice 54 completely.

The ventilator-resuscitator device 12 may further comprise a pressure sensor (not illustrated) configured to measure the pressure of gas exiting therefrom, i.e., downstream of the high-pressure side 44 of the compressor 18. Accordingly, the pressure sensor may be in fluid communication with the measurement outlet 52. As the measurement outlet 52 is in isobaric fluid communication (i.e., in fluid communication such that both are at the same pressure) with the outlet nipple 48, the pressure sensor indicates the pressure of gas delivered to the patient via the mask 14.

In addition to the above, the outlet tube 46 may comprise other suitable elements, such as a safety valve 56, etc., without departing from the scope of the present disclosure and claims.

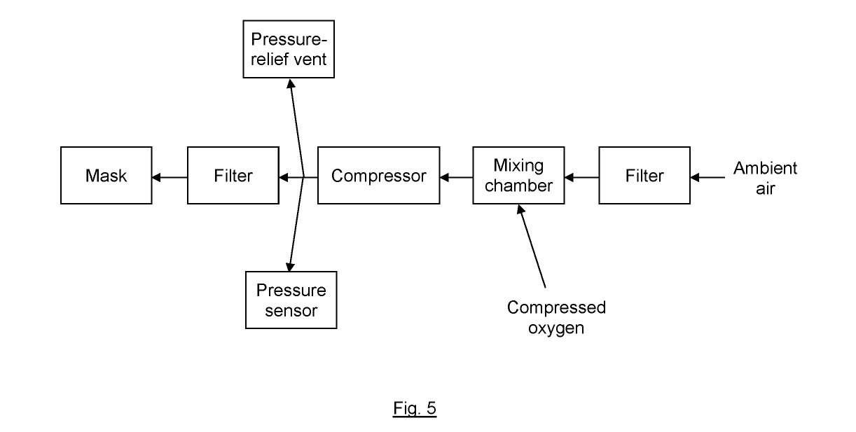

As illustrated in FIG. 5, the ventilator-resuscitator device 12 may define a fluid path through the various elements thereof Thus, ambient air (through a filter) and compressed oxygen flow to the mixing chamber to form a gas mixture. The gas mixture is drawn into the compressor, where it is pressurized. It exits the compressor and flows through a filter to the mask. Between the filter and the mask, the compressed gas mixture is measured by a pressure sensor, and its pressure may be regulated by the pressure-relief vent.

As suggested above, the controller 22 may be configured to regulate the pressure of the gas delivered to the patient by concurrently controlling the power level of the compressor 18, and the size of the orifice 54 of the pressure-relief vent 50. By adjusting these two parameters, the pressure of the pressurized gas supplied by the ventilator-resuscitator device 12 may better simulate a desired pressure profile (i.e., pressure waveform) over time in a patient's lungs than could be done using only one of the parameters.

As several factors may contribute to the pressure of the gas (e.g., the pressure of the oxygen, atmospheric pressure, the increase in pressure contributed by the compressor 18, the decrease in pressure owing to the orifice 54 of the pressure-relief vent 50, etc.), it may be more practical to measure the pressure of gas exiting the outlet nipple 48 and delivered to the patient than to calculate it. Thus, the controller 22 may be configured to perform a calibration, e.g., upon commencement of ventilation. According to some examples, the calibration may include adjusting parameters of the ventilator-resuscitator device 12 in predetermined ways (the parameters may include, but are not limited to, the power level of the compressor 18 and the size of the orifice 54 of the pressure-relief vent 50), and measuring the pressure of the gas for several combinations of the parameters. The controller 22 may then operate the elements of the ventilator-resuscitator device 12 in order to achieve desired pressures during ventilation.

In addition, the controller 22 may be configured to alert a user when an unexpected event occurs. According to one example, the controller 22 may be configured to activate an alarm if an unexpected, e.g., sudden, increase in pressure is detected. Such an increase may indicate that a tube connecting the ventilator-resuscitator device 12 to the mask has developed a kink, preventing the patient to receive the gas mixture at a sufficient pressure.

According to another example, the controller 22 may be configured to activate an alarm if an unexpected, e.g., sudden, decrease in pressure is detected. Such an increase may indicate that the mask has fallen from, or has otherwise disengaged from, the patient's face.

The alarm may be an audible alarm. Accordingly, the controller may comprise a suitable element for producing the audible alarm. Alternatively, the ventilator-resuscitator device 12 may comprise a dedicated element (not illustrated), whose operation is directed by the controller 22, and which is configured to produce a sound.

The controller 22 may be configured to cyclically regulate the pressure of said pressurized gas between two pressures at a predetermined rate, for example to simulate breathing in an attempt to stimulate a patient to breathe on his own. Accordingly, the predetermined rate may match that of an appropriate breathing rate for the patient. According to some modifications, the controller 22 is configured to issue an audible sound at a predetermined rate. This allows a doctor to manually adjust the pressure (for example by alternatingly covering and uncovering an aperture in or near the mask), with the controller 22 facilitating the doctor to do so at a predetermined rate.

Those skilled in the art to which this invention pertains will readily appreciate that numerous changes, variations and modifications can be made without departing from the scope of the invention mutatis mutandis.

Technical and scientific terms used herein should have the same meaning as commonly understood by one of ordinary skill in the art to which the disclosure pertains. Nevertheless, it is expected that during the life of a patent maturing from this application many relevant systems and methods will be developed. Accordingly, the scope of the terms such as computing unit, network, display, memory, server and the like are intended to include all such new technologies a priori.

The terms “comprises”, “comprising”, “includes”, “including”, “having” and their conjugates mean “including but not limited to” and indicate that the components listed are included, but not generally to the exclusion of other components. Such terms encompass the terms “consisting of” and “consisting essentially of”.

The phrase “consisting essentially of” means that the composition or method may include additional ingredients and/or steps, but only if the additional ingredients and/or steps do not materially alter the basic and novel characteristics of the composition or method.

As used herein, the singular form “a”, “an” and “the” may include plural references unless the context clearly dictates otherwise. For example, the term “a compound” or “at least one compound” may include a plurality of compounds, including mixtures thereof.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination or as suitable in any other described to embodiment of the disclosure. Certain features described in the context of various embodiments are not to be considered essential features of those embodiments, unless the embodiment is inoperative without those elements.

Although the disclosure has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the disclosure.

All publications, patents and patent applications mentioned in this disclosure are herein incorporated in their entirety by reference into the disclosure, to the same extent as if each individual publication, patent or patent application was specifically and individually indicated to be incorporated herein by reference. In addition, citation or identification of any reference in this application shall not be construed as an admission that such reference is available as prior art to the present disclosure. To the extent that section headings are used, they should not be construed as necessarily limiting.

1-47. (canceled)

48. A ventilator-resuscitator device comprising:
a mixing chamber having an air inlet, an oxygen inlet, and an outlet;
a compressor configured to operate at two or more power levels and to increase, by a different amount at each power level, the pressure of a gas flowing between a low-pressure side thereof and a high-pressure side thereof, thereby producing a pressurized gas, said low-pressure side being in fluid communication with the outlet of said mixing chamber;
a controllable pressure-relief vent comprising an orifice configured to bring said high-pressure side of the compressor into fluid communication with the atmosphere; and
a controller configured to direct operation thereof;
wherein said controller is configured to regulate the pressure of said pressurized gas by selectively operating said compressor at one of its power levels.

49. The ventilator-resuscitator device according to claim 48, wherein said controller is further configured to regulate the pressure of said pressurized gas by operating said controllable pressure-relief vent to selectively change the size of said orifice.

50. The ventilator-resuscitator device according to claim 49, wherein said controller is configured to regulate the pressure of said pressurized gas by concurrently operating:
said controller at one of its power levels; and
said controllable pressure-relief vent to change the size of said orifice.

51. The ventilator-resuscitator device according to claim 48, configured to selectively close said orifice completely.

52. The ventilator-resuscitator device according to claim 48, wherein said controllable pressure-relief vent comprises one or more solenoids configured to regulate the size of said orifice.

53. The ventilator-resuscitator device according to claim 48, further comprising a pressure sensor configured to measure the pressure of gas exiting therefrom.

54. The ventilator-resuscitator device according to claim 53, said controller being configured to perform a calibration based on the pressure measured by said pressure sensor.

55. The ventilator-resuscitator device according to claim 53, wherein said controller is configured to activate an alarm when an unexpected increase in pressure is measured by said pressure sensor.

56. The ventilator-resuscitator device according to claim 53, wherein said controller is configured to activate an alarm when an unexpected decrease in pressure is measured by said pressure sensor.

57. The ventilator-resuscitator device according to claim 48, wherein said controller is configured to cyclically regulate the pressure of said pressurized gas between two pressures at a predetermined rate.

58. The ventilator-resuscitator device according to claim 48, wherein said controller is configured to emit an audible sound at a predetermined rate.

59. The ventilator-resuscitator device according to claim 48, wherein said compressor comprises a variable-speed drive.

60. The ventilator-resuscitator device according to claim 48, comprising a battery configured to supply electrical power necessary for its operation.

61. The ventilator-resuscitator device according to claim 48, wherein said oxygen inlet comprises an oxygen inlet nipple configured for attachment thereto of a flexible tube.

62. The ventilator-resuscitator device according to claim 48, further comprising an outlet nipple, configured for attachment to a flexible tube, in fluid communication with the high-pressure side of said compressor.

63. The ventilator-resuscitator device according to claim 62, wherein said controllable pressure-relief vent is located in fluid communication between the high-pressure side of said compressor and said outlet nipple.

64. A ventilator-resuscitator system comprising a ventilator-resuscitator device and a mask, the ventilator-resuscitator device comprising:
a mixing chamber having an air inlet, an oxygen inlet, and an outlet;
a compressor configured to operate at two or more power levels and to increase, by a different amount at each power level, the pressure of a gas flowing between a low-pressure side thereof and a high-pressure side thereof, said low-pressure side being in fluid communication with the outlet of said mixing chamber;
a controllable pressure-relief vent comprising an orifice configured to bring said high-pressure side of the compressor into fluid communication with the atmosphere; and
a controller configured to direct operation thereof;
said mask being configured to:
be brought into fluid communication with the high-pressure side of said compressor for receipt therefrom of a pressurized gas; and
be fitted to the face of a patient for delivery thereto of said pressurized gas;
wherein said controller is configured to regulate the pressure of said pressurized gas by selectively operating said compressor at one of its power levels.

65. A method of ventilating a patient, the method comprising providing a ventilator-resuscitator device, said ventilator-resuscitator device comprising:
a mixing chamber for producing an oxygen-air mixture and having an air inlet, an oxygen inlet, and an outlet;
a compressor configured to operate at two or more power levels and to increase, by a different amount at each power level, the pressure of a said oxygen-air mixture flowing between a low-pressure side thereof and a high-pressure side thereof, said low-pressure side being in fluid communication with the outlet of said mixing chamber to receive therefrom the oxygen-air mixture;
a controllable pressure-relief vent comprising an orifice configured to bring said high-pressure side of the compressor into fluid communication with the atmosphere; and
a controller configured to direct operation thereof;
said method further comprising:
connecting a mask to said ventilator-resuscitator device to receive the compressed oxygen-air mixture therefrom;
fitting the mask to said patient to deliver said oxygen-air mixture from the ventilator-resuscitator device thereto;
operating said ventilator-resuscitator system to deliver a pressurized oxygen-air mixture to said patient; and
regulating the pressure of said oxygen-air mixture between two pressures by selectively operating said compressor at two of its power levels.

66. The method according to claim 65, further comprising regulating the pressure of said oxygen-air mixture by concurrently operating:
said controller at two of its power levels; and
said controllable pressure-relief vent to change the size of said orifice.

67. The method according to claim 66, further comprising performing a calibration based on the pressure.
